(19) United States
(12) Patent Application Publication
(10) Pub. No.: US 2021/0096354 A1
(43) Pub. Date: Apr. 1, 2021

(54) IMAGE PICKUP APPARATUS, ENDOSCOPE APPARATUS, AND METHOD OF ELECTRICAL CONNECTION TO IMAGE PICKUP MODULE IN IMAGE PICKUP APPARATUS

(71) Applicant: OLYMPUS CORPORATION, Tokyo (JP)

(72) Inventors: Keiichi KOBAYASHI, Sagamihara-shi (JP); Takatoshi IGARASHI, Tokyo (JP); Takahiro SHIMOHATA, Shiojiri-shi (JP)

(73) Assignee: OLYMPUS CORPORATION, Tokyo (JP)

(21) Appl. No.: 17/028,015

(22) Filed: Sep. 22, 2020

Related U.S. Application Data
(63) Continuation of application No. PCT/JP2018/012038, filed on Mar. 26, 2018.

(51) Int. Cl. G02B 23/24

(52) U.S. Cl. CPC G02B 23/2484; A61B 1/051; G02B 23/2492

(57) ABSTRACT

An image pickup apparatus includes an image pickup module in which an image pickup device is laminated, a relay board having a plurality of flying leads which are electrically connected to a proximal end face of the image pickup module through ultrasonic wire bonding, and a plurality of cables electrically connected to a plurality of connection lands of the relay board, and a bent portion is formed on the relay board so that the plurality of cables are fitted within a projection surface of the proximal end face of the image pickup module.

### CROSS REFERENCE TO RELATED APPLICATION

This application is a continuation application of PCT/JP2018/012038 filed on Mar. 26, 2018, the entire contents of which are incorporated herein by this reference.

### BACKGROUND OF THE INVENTION

### 1. Field of the Invention

The present invention relates to an image pickup apparatus incorporated in an endoscope, an endoscope apparatus provided with the image pickup apparatus and a method of electrical connection to an image pickup module in the image pickup apparatus.

### 2. Description of the Related Art

To observe areas that are difficult to observe such as an inside of a living body or an inside of a structure, electronic endoscopes that can be introduced from outside into the living body or the structure and provided with an image pickup apparatus or the like to pick up an optical image are being used, for example, in a medical field or industrial field.

The image pickup apparatus of the electronic endoscope is provided with an objective lens configured to form an object image and an image sensor disposed on an image-forming plane of the objective lens. Such an image sensor is mounted with a semiconductor device.

For example, Japanese Patent Application Laid-Open Publication No. 2010-263020 discloses an endoscope apparatus having a configuration in which a wiring board with a cable is connected to an image pickup module where an image pickup device is laminated on a surface of a substrate.

### SUMMARY OF THE INVENTION

An image pickup apparatus according to one aspect of the present invention includes an image pickup module in which an image pickup device is laminated, a relay board having a plurality of flying leads which are electrically connected to a proximal end face of the image pickup module through ultrasonic wire bonding, and a plurality of cables electrically connected to a plurality of connection lands of the relay board, in which a bending portion is formed on the relay board so that the plurality of cables are fitted within a projection surface of the proximal end face of the image pickup module and the bending portion is bent so as to have an arched cross section.

An image pickup apparatus according to another aspect of the present invention includes an image pickup module in which an image pickup device is laminated, a relay board having a plurality of flying leads which are electrically connected to a proximal end face of the image pickup module through ultrasonic wire bonding, and a plurality of cables electrically connected to a plurality of connection lands of the relay board, in which the relay board includes a small-width portion on a distal end side connected to the image pickup module, the bending portion on a proximal end side of the small-width portion and a wide, large-width portion provided with the plurality of connection lands, the bending portion is formed on the relay board so that the plurality of cables are fitted within a projection surface of the proximal end face of the image pickup module, a large-diameter cable of the plurality of cables is a coaxial cable and the bending portion along an external conductor of the coaxial cable is formed at the large-width portion.

An endoscope apparatus according to one aspect of the present invention includes an image pickup apparatus including an image pickup module in which an image pickup devices is laminated, a relay board having a plurality of flying leads which are electrically connected to a proximal end face of the image pickup module through ultrasonic wire bonding, and a plurality of cables electrically connected to a plurality of connection lands of the relay board, in which a bending portion is formed on the relay board so that the plurality of cables are fitted within a projection surface of the proximal end face of the image pickup module and the bending portion is bent so as to have an arched cross section, and an insertion portion provided with a distal end portion in which the image pickup apparatus is incorporated.

A method of electrical connection to an image pickup module in an image pickup apparatus according to one aspect of the present invention is a method of electrical connection to an image pickup module in an image pickup apparatus, the image pickup apparatus including an image pickup module in which an image pickup devices is laminated, a relay board having a plurality of flying leads which are electrically connected to a proximal end face of the image pickup module through ultrasonic wire bonding, and a plurality of cables electrically connected to a plurality of connection lands of the relay board, in which a bending portion is formed on the relay board so that the plurality of cables are fitted within a projection surface of the proximal end face of the image pickup module and the bending portion is bent so as to have an arched cross section, the method including soldering the plurality of cables to the plurality of connection lands of the relay board, ultrasonic wire bonding the plurality of flying leads of the relay board to a plurality of terminals provided on the proximal end face of the image pickup module, and forming the bending member on the relay board so that the plurality of cables are fitted within the projection surface of the proximal end face of the image pickup module.

### BRIEF DESCRIPTION OF THE DRAWINGS

FIG. 1 is a perspective view illustrating a configuration of an endoscope system according to one aspect of the present invention;

FIG. 2 is a perspective view illustrating a configuration of an image pickup apparatus according to one aspect of the present invention;

FIG. 3 is a side view illustrating the configuration of the image pickup apparatus according to one aspect of the present invention;

FIG. 4 is a cross-sectional view illustrating a rear side of the image pickup apparatus according to one aspect of the present invention;

FIG. 5 is an exploded perspective view illustrating an image pickup cable and various electric cables electrically connected to a relay board of the image pickup apparatus according to one aspect of the present invention;

FIG. 6 is a perspective view illustrating the image pickup cable and various electric cables electrically connected to the relay board of the image pickup apparatus according to one aspect of the present invention;

FIG. 7 is a perspective view illustrating the relay board electrically connected to an image pickup module of the image pickup apparatus according to one aspect of the present invention,

FIG. 8 is a perspective view illustrating the relay board bent in the image pickup apparatus according to one aspect of the present invention;

FIG. 9 is an exploded perspective view illustrating the image pickup cable and the various electric cables electrically connected to a relay board according to a first modification of the image pickup apparatus according to one aspect of the present invention;

FIG. 10 is a cross-sectional view illustrating a rear side of the image pickup apparatus according to the first modification of the image pickup apparatus according to one aspect of the present invention;

FIG. 11 is a perspective view illustrating a configuration of a relay board according to another aspect of the first modification of the image pickup apparatus according to one aspect of the present invention;

FIG. 12 is a cross-sectional view illustrating a rear side of the image pickup apparatus according to the other aspect of the first modification of the image pickup apparatus according to one aspect of the present invention;

FIG. 13 is an exploded perspective view illustrating a configuration of a jig together with an image pickup cable and various electric cables electrically connected to the relay board according to a second modification of the image pickup apparatus according to one aspect of the present invention;

FIG. 14 is a perspective view illustrating the relay board held to the jig according to the second modification of the image pickup apparatus according to one aspect of the present invention;

FIG. 15 is a partial cross-sectional view illustrating the relay board held to the jig according to the second modification of the image pickup apparatus according to one aspect of the present invention; and

FIG. 16 is a side view illustrating a configuration of the image pickup apparatus according to the second modification of the image pickup apparatus according to one aspect of the present invention.

### DETAILED DESCRIPTION OF THE PREFERRED EMBODIMENT

Hereinafter, a preferred embodiment of the present invention will be described with reference to the accompanying drawings.

Note that in the respective drawings used in following description, a scale is made to differ for each component so that each component is illustrated in size recognizable in each drawing. However, the present invention is not limited only to quantities of components, shapes of the components, a size ratio among the components and relative positional relationships among the components described in the drawings. In the following description, there are cases where an up-down direction as viewed in the plane of the drawing is described as upper and lower parts of the components.

First, an endoscope apparatus provided with an image pickup apparatus 1 including an image pickup module according to one aspect of the present invention will be described hereinafter based on the accompanying drawings.

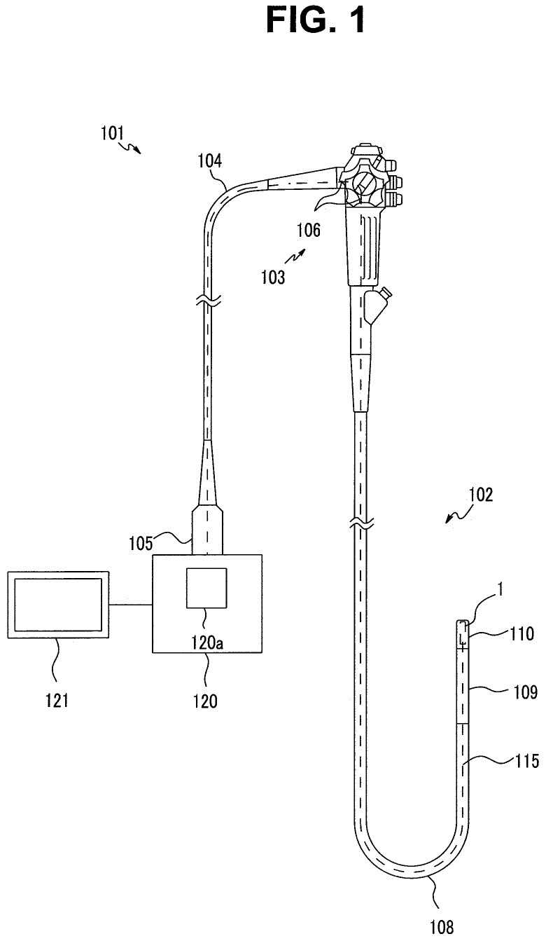

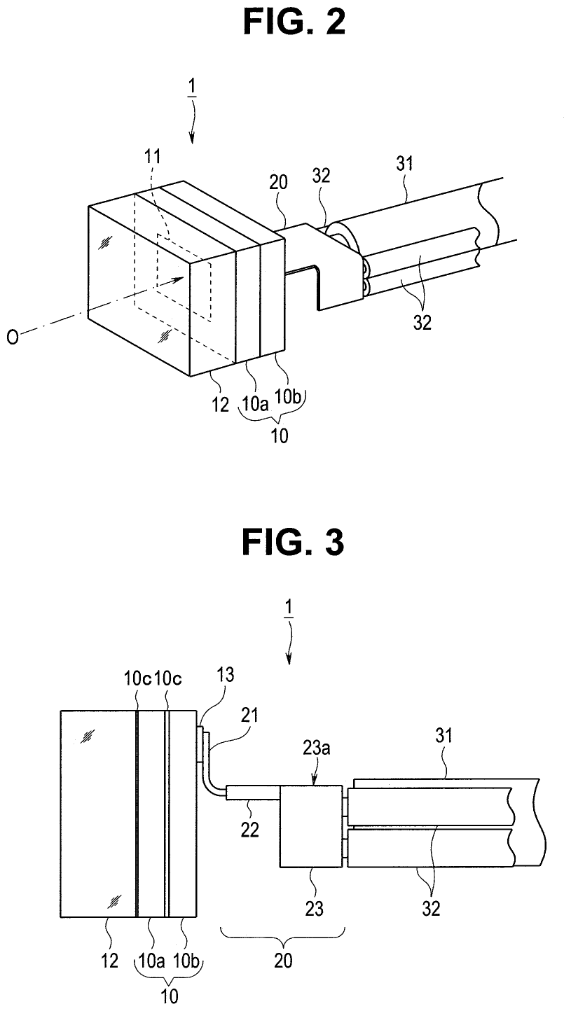

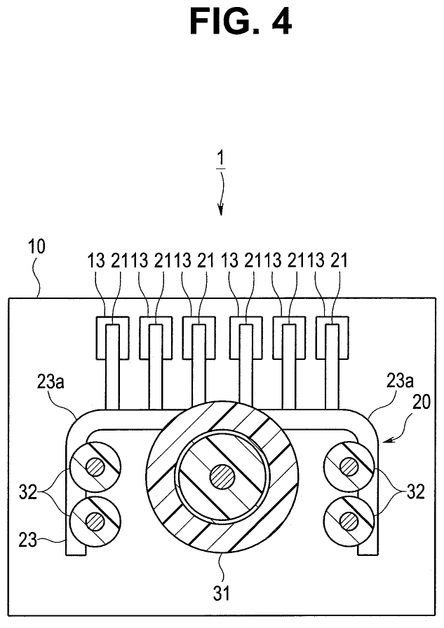

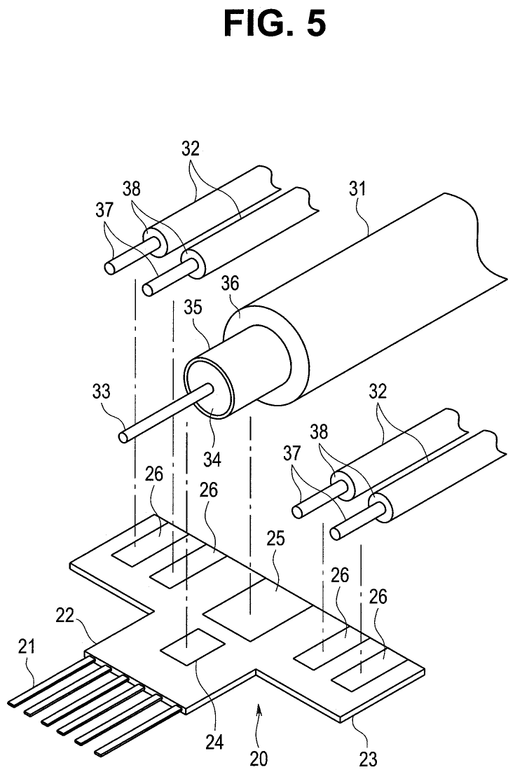

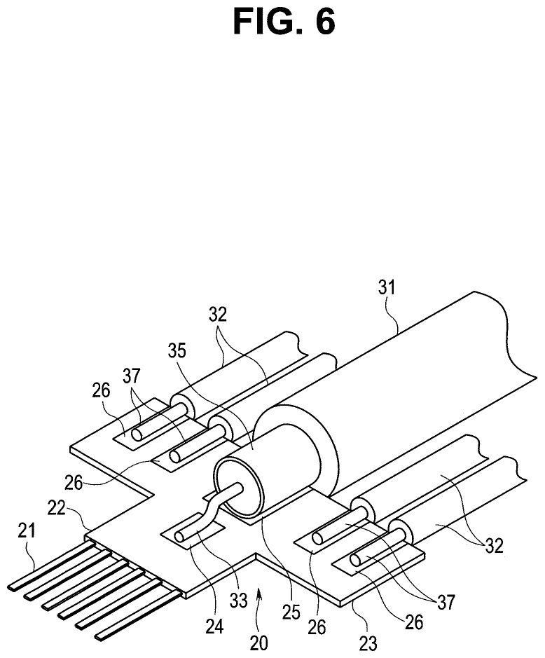

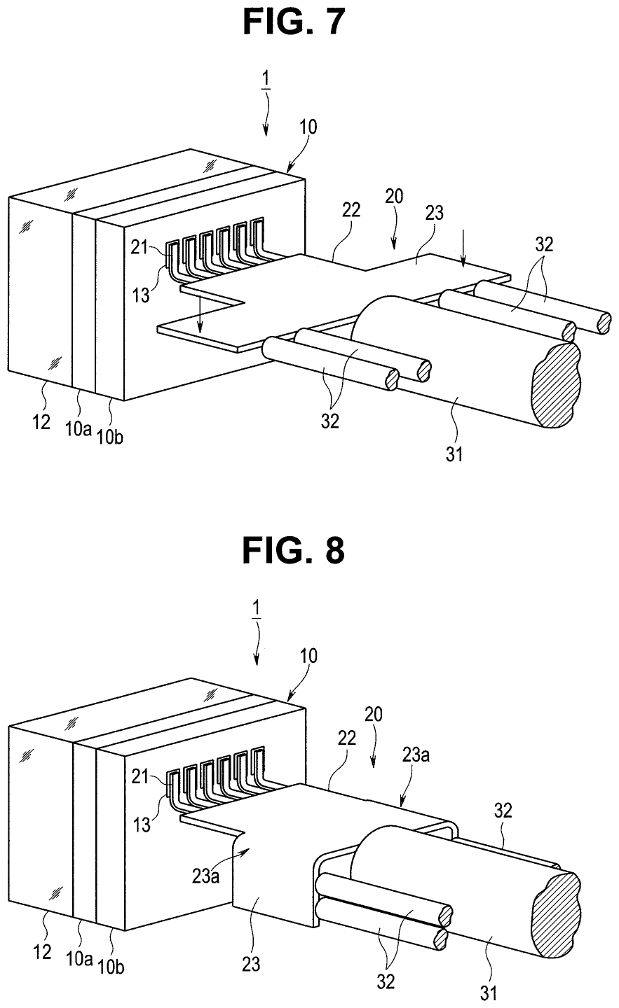

Note that FIG. 1 is a perspective view illustrating a configuration of an endoscope system according to one aspect of the present invention, FIG. 2 is a perspective view illustrating a configuration of an image pickup apparatus, FIG. 3 is a side view illustrating a configuration of the image pickup apparatus, FIG. 4 is a cross-sectional view illustrating a rear side of the image pickup apparatus, FIG. 5 is an exploded perspective view illustrating an image pickup cable and various electric cables electrically connected to a relay board, FIG. 6 is a perspective view illustrating the image pickup cable and various electric cables electrically connected to the relay board, FIG. 7 is a perspective view illustrating the relay board electrically connected to the image pickup module, and FIG. 8 is a perspective view illustrating the bent relay board.

First, an example of a configuration of an endoscope apparatus provided with an image pickup apparatus 1 according to the present invention will be described with reference to FIG. 1.

An endoscope apparatus (hereinafter abbreviated as an “endoscope”) 101 as an endoscope system of the present embodiment can be introduced into a subject such as a human body and is configured to optically pick up an image of a predetermined observation region in the subject.

Note that the subject into which the endoscope 101 is introduced is not limited to the human body, but may be another living body or an artificial object such as a machine or a building.

The endoscope 101 is mainly constructed of an insertion portion 102 to be introduced into the subject, an operation portion 103 located at a proximal end of the insertion portion 102 and a universal cord 104 that extends from a side part of the operation portion 103.

The insertion portion 102 is constructed by consecutively connecting a distal end portion 110 disposed at a distal end, a bendable bending section 109 disposed on a proximal end side of the distal end portion 110 and a flexible tube portion 108 having flexibility disposed on a proximal end side of the bending section 109 and connected to a distal end side of the operation portion 103.

The endoscope 101 here is a bronchoscope or the like, the diameter of the insertion portion 102 of which is particularly small. Note that the endoscope 101 may be of a type with the insertion portion 102 without any flexible region, a so-called rigid endoscope.

The distal end portion 110 is provided with an image pickup apparatus 1 including an image pickup module 10, which will be described later. The operation portion 103 is also provided with angle operation knobs 106 for operating bending of the bending section 109.

An endoscope connector 105 connected to an external apparatus 120 is provided at a proximal end portion of the universal cord 104. The external apparatus 120 to which the endoscope connector 105 is connected is connected to an image display section 121 such as a monitor via a cable.

The endoscope 101 also includes a composite cable 115 inserted in the universal cord 104, the operation portion 103 and the insertion portion 102, and an optical fiber bundle (not shown) configured to transmit illumination light from a light source section provided in the external apparatus 120.

The composite cable 115 is configured to electrically connect the endoscope connector 105 with the image pickup apparatus 1. When the endoscope connector 105 is connected to the external apparatus 120, the image pickup apparatus 1 is electrically connected to the external apparatus 120 via the composite cable 115.

Via the composite cable 115, electric power is supplied from the external apparatus 120 to the image pickup apparatus 1 and the external apparatus 120 communicates with the image pickup apparatus 1.

The external apparatus 120 is provided with an image processing section 120a. The image processing section 120a generates a video signal based on an image pickup device output signal outputted from the image pickup apparatus 1 and outputs the signal to the image display section 121. In other words, in the present embodiment, an optical image (endoscopic image) picked up by the image pickup apparatus 1 is displayed on the image display section 121 as a video.

Note that the endoscope 101 is not limited to the configuration in which the endoscope 101 is connected to the external apparatus 120 or the image display section 121, but the endoscope 101 may also have a configuration including a part or a whole of the image processing section or the monitor.

The optical fiber bundle is configured to transmit light emitted from the light source section of the external apparatus 120 to an illumination window as an illumination light emitting section of the distal end portion 110. Furthermore, the light source section may be disposed at the operation portion 103 or the distal end portion 110 of the endoscope 101.

Next, a configuration of the image pickup module 10 provided in the image pickup apparatus 1 provided at the distal end portion 110 will be described. Note that in the following description, a direction on an object side from the image pickup module 10 toward the subject (leftward in each drawing) may be referred to as a “distal end” or “forward” and an opposite direction on an image side may be referred to as a “proximal end” or “rearward.”

As illustrated in FIG. 2, the image pickup apparatus 1 of the present embodiment is provided with an image pickup module 10 of a CSP (chip size package) constructed of a cover glass 12 provided on a front side and an integrated circuit (IC chip) such as an image pickup device 10a and a drive circuit chip 10b pasted by an adhesive 10c and laminated together.

The image pickup module 10 is electrically connected to an image pickup cable 31, which is a coaxial cable on the order of 0.2 mm to 0.5 mm, a power cable on the order of 0.1 mm to 0.15 mm and various electric cables 32 such as a ground wire via a relay board 20, which is an FPC (flexible printed circuit board) on the proximal end side.

Note that a light-receiving section 11 configured to receive light of a subject image having a photographing optical axis O is disposed on a front side of the image pickup device 10a of the image pickup module 10. The image pickup device 10a is an image sensor chip such as a CCD or a CMOS, and the aforementioned cover glass 12 is disposed at a front portion provided with the light-receiving section 11.

The relay board 20 is provided with a plurality of flying leads 21 on a distal end side as a connection section electrically connected to the image pickup module 10, a surface of which constitutes a substantially T-shaped belt-like FPC including a small-width section 22 on a distal end side and a large-width section 23 on a proximal end side (see FIG. 5). In the relay board 20, a plurality of (six here) flying leads 21 extend in parallel from an edge of the small-width section 22.

As illustrated in FIG. 3, respective end portions of the plurality of flying leads 21 are ultrasonically wire bonded to a plurality of (six here) terminals 13 provided in parallel on the proximal end face, which is a rear face of the image pickup module 10 and are bent vertically upward so as to stand upright with respect to a horizontal direction along the surface of the small-width section 22.

As illustrated in FIG. 4, the large-width section 23 of the relay board 20 is bent in a vertically downward direction opposite to the vertically upward bending direction of the plurality of flying leads 21 so as to fit within a projection surface of the image pickup module 10, thus forming bent portions 23a.

Here, an electrical connection procedure (connection method) of connections of the image pickup cable 31 and the various electric cables 32 to the relay board 20 and a connection of the relay board 20 to the image pickup module 10 will be described.

As illustrated in FIG. 5 and FIG. 6, a plurality of connection lands 24, 25 and 26 are provided on one side of the relay board 20 and the image pickup cable 31 and the various electric cables 32 are electrically connected to the connection lands 24, 25 and 26 by solder. The plurality of connection lands 24, 25 and 26 here are provided on the same side of the relay board 20.

More specifically, an internal conductor 33 stripped of an insulator 34 of the image pickup cable 31 is soldered to the connection land 24 on a central distal end side and an external conductor 35 stripped of a coating sheath 36 is soldered to the connection land 25 on a central proximal end side.

Core wires 37 stripped of outer layers 38 of the various electric cables 32 are soldered to the connection lands 26 on both side portions of the large-width section 23.

In other words, the image pickup cable 31 of a largest diameter is electrically connected to a center of the relay board 20 and the various electric cables 32 of a small diameter are electrically connected to both side portions.

Thus, the connection lands 24 and 25 for soldering the image pickup cable 31 are provided at the center of the relay board 20 and the connection lands 26 for soldering the various electric cables 32 are provided on the wide large-width section 23 on both sides of the connection lands 24 and 25, and so sufficient separation distances are provided for the connection lands 24, 25 and 26.

Such a configuration makes it possible to suppress thermal influences such as melting on the other already connected soldered parts at the time of soldering the image pickup cable 31 and the various electric cables 32 and prevent insufficient connections or the like.

Next, as illustrated in FIG. 7, the plurality of flying leads 21 are electrically connected through ultrasonic wire bonding to the plurality of terminals 13 provided on the proximal end face of the image pickup module 10 and are bent upward.

The plurality of flying leads 21 are bent upward, connected to the plurality of terminals 13, and then both side portions of the large-width section 23 to which the various electric cables 32 are connected are bent downward to form the bent portions 23a.

In this way, the image pickup module 10, the image pickup cable 31 and the various electric cables 32 are electrically connected together via the relay board 20 so that the relay board 20, the image pickup cable 31 and the various electric cables 32 are fitted within the projection surface of the image pickup module 10.

Thus, in the image pickup apparatus 1 of the present embodiment, the relay board 20 in a flat state, with which the image pickup cable 31 and the various electric cables 32 are electrically connected is electrically connected to the image pickup module 10, which facilitates positioning and handling or the like during manufacturing.

The image pickup cable 31 and the various electric cables 32 are electrically connected to the relay board 20 by solder in advance and the plurality of flying leads 21 of the relay board 20 are ultrasonically wire bonded to the plurality of terminals 13 of the image pickup module 10, and it is thereby possible to prevent thermal load due to soldering to the image pickup module 10 and avoid damage to the laminated connection part of the image pickup module 10.

Furthermore, by forming the bent portions 23a on the relay board 20 so that the image pickup cable 31 and the various electric cables 32 are fitted within the projection surface of the image pickup module 10, it is possible to prevent the outer diameter from increasing.

As described so far, the image pickup apparatus 1 disposed in the endoscope 101 of the present embodiment has a configuration that can avoid thermal load from being added to the image pickup module 10 and thereby prevent damage and prevent the outer diameter from increasing.

(First Modification)

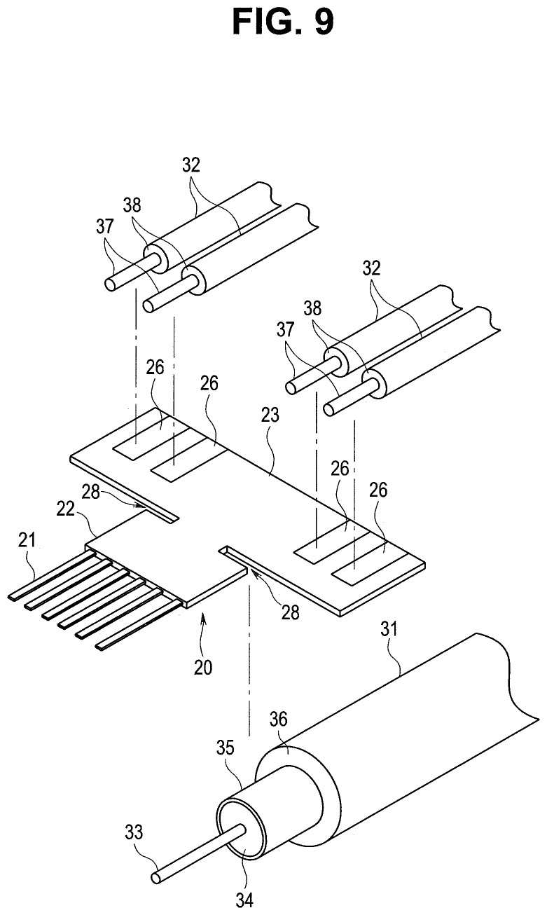

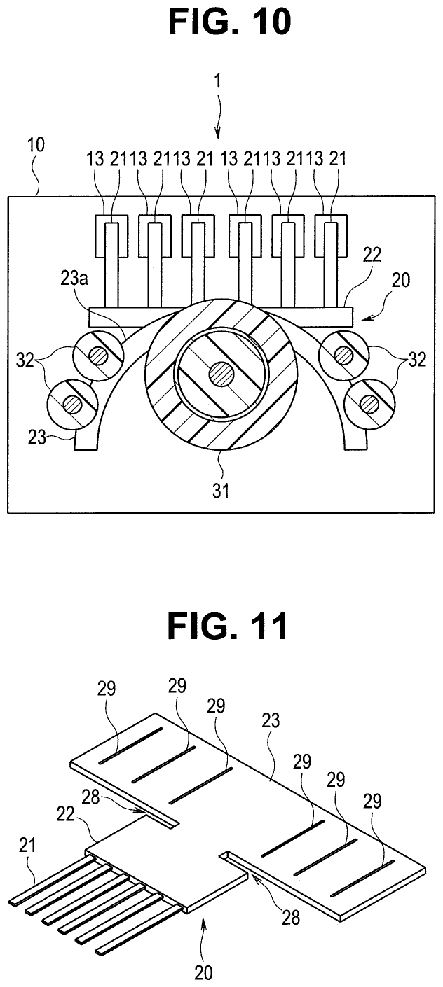

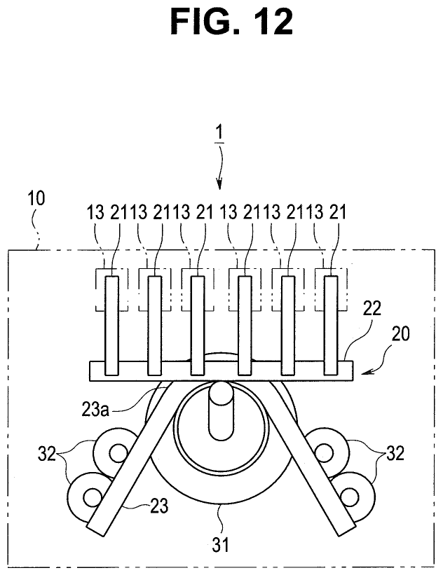

FIG. 9 is an exploded perspective view illustrating the image pickup cable and the various electric cables electrically connected to the relay board according to a first modification, FIG. 10 is a cross-sectional view illustrating a rear side of the image pickup apparatus according to the first modification, FIG. 11 is a perspective view illustrating a configuration of the relay board according to another aspect of the first modification, and FIG. 12 is a cross-sectional view illustrating a rear side of the image pickup apparatus according to the other aspect of the first modification.

As illustrated in FIG. 9, notch portions 28 that constitute a constricted portion from both sides of a boundary portion between the small-width section 22 and the large-width section 23 may be formed and the large-width section 23 may be formed into the bent portions 23a having an arched cross section as illustrated in FIG. 10 in the relay board 20 according to the present modification.

Note that the image pickup cable 31 is electrically connected to one side, which constitutes a curved inside of the relay board 20, and the various electric cables 32 are electrically connected to the other side, which constitutes a curved outside of the relay board 20, and the large-width section 23 is bent so as to have an arched cross section so that the image pickup cable 31 and the various electric cables 32 are fitted within the projection surface of the image pickup module 10.

In the relay board 20 of the present modification, the image pickup cable 31 and the various electric cables 32 can be fitted within the projection surface of the image pickup module 10 with lower load than the load with which the large-width section 23 is bent as in the case of the aforementioned embodiment.

By connecting the large-diameter image pickup cable 31 to the curved inside of the relay board 20 and connecting the small-diameter various electric cables 32 to the curved outside of the relay board 20, the parts connected by solder are further separated from each other so as not to be affected by heat.

Note that as illustrated in FIG. 11, the relay board 20 may also have a configuration with a plurality of slits 29 disposed in a front-rear direction in the large-width section 23 to facilitate bending.

Moreover, as illustrated in FIG. 12, the large-width section 23 of the relay board 20 may also be formed into the arched bent portions 23a along the external conductor 35 of the image pickup cable 31 so that the image pickup cable 31 and the various electric cables 32 are fitted within the projection surface of the image pickup module 10.

(Second Modification)

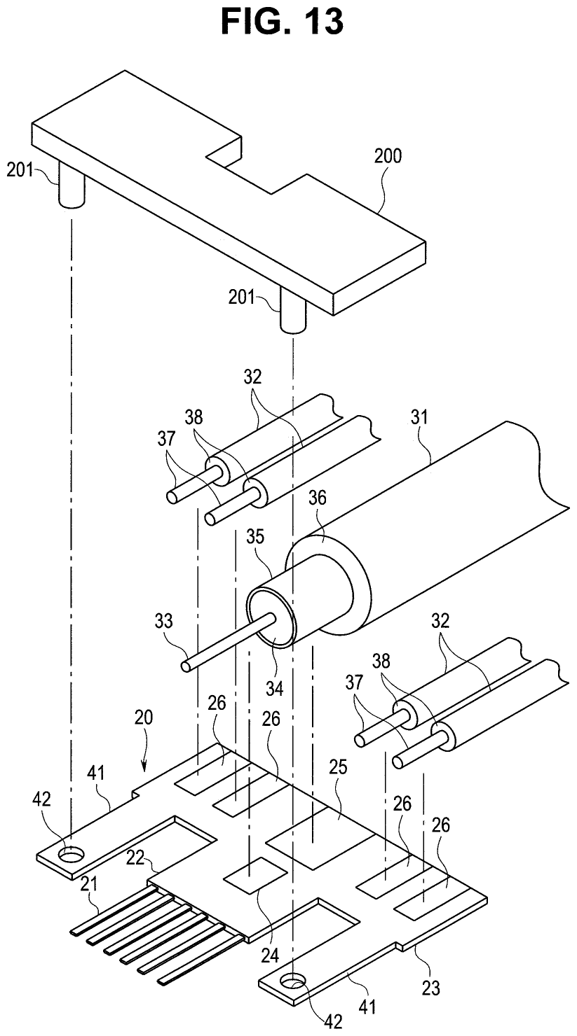

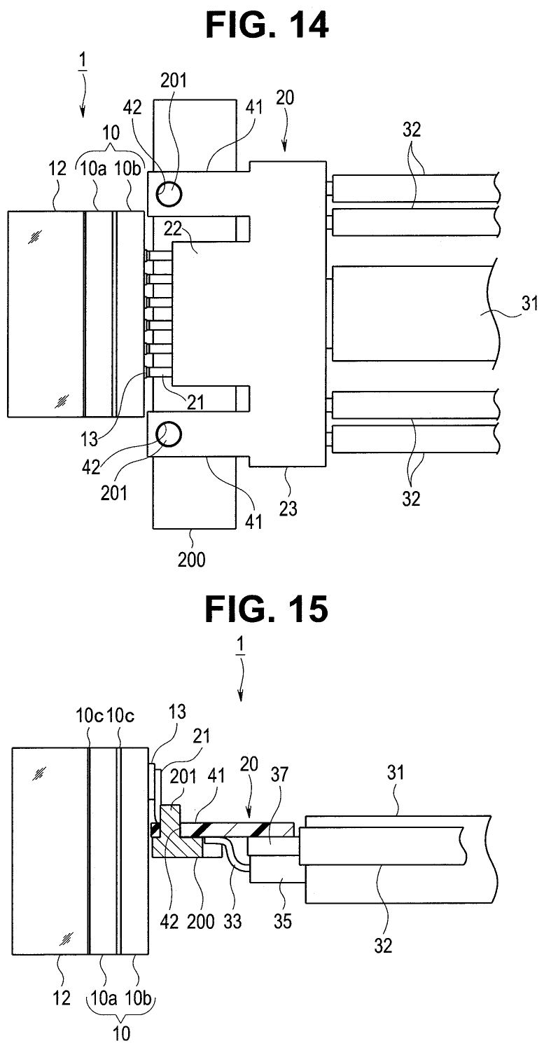

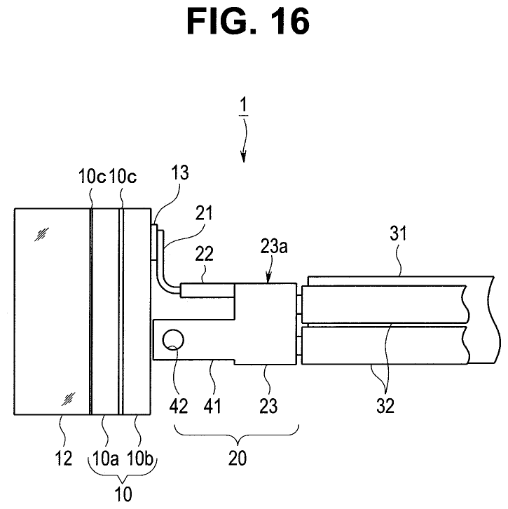

FIG. 13 is an exploded perspective view illustrating a configuration of a jig together with an image pickup cable and various electric cables electrically connected to the relay board according to a second modification, FIG. 14 is a perspective view illustrating the relay board held to the jig according to the second modification, FIG. 15 is a partial cross-sectional view illustrating the relay board held to the jig according to the second modification, and FIG. 16 is a side view illustrating a configuration of the image pickup apparatus according to the second modification.

As illustrated in FIG. 13, the relay board 20 according to the present modification has a configuration in which two arm portions 41 extending from both side portions of the large-width section 23 toward the distal end side are provided and hole portions 42 through which protrusion portions 201 of a jig 200 (see FIG. 14) are passed are perforated in the two arm portions 41. The two arm portions 41 are slightly longer than the small-width section 22 and have lengths to project to the distal end side.

As illustrated in FIG. 14 and FIG. 15, when the plurality of flying leads 21 of the relay board 20 are ultrasonically wire bonded to the plurality of terminals 13 of the image pickup module 10, the two protrusion portions 201 of the jig 200 are inserted into the hole portions 42 of the arm portions 41, and the relay board 20 is grasped by the jig 200 so as to prevent stress caused by the connected image pickup cable 31 and various electric cables 32 from transmitting to the plurality of flying leads 21. It is thereby possible to reduce the load on the connection portions that electrically connect the plurality of flying leads 21 to the plurality of terminals 13.

Furthermore, as illustrated in FIG. 16, when the bent portions 23a are formed in the relay board 20, the two arm portions 41 come into contact with the proximal end face of the image pickup module 10 and it is possible to prevent the relay board 20 from collapsing under the weights of the image pickup cable 31 and the various electric cables 32.

In the above described embodiment and respective modifications, the respective components may be combined. In other words, the invention described in the above described embodiment is not limited to the embodiment and the modifications, but can be implemented by making various modifications without departing from the spirit and scope of the invention in the implementation phase. Furthermore, the above-described embodiment includes inventions in various phases and various inventions can be extracted according to appropriate combinations in a plurality of disclosed configuration requirements.

For instance, even when some configuration requirements are deleted from all the configuration requirements illustrated in the embodiment, configurations from which these configuration requirements have been deleted can be extracted as inventions as long as the described problems can be solved and the described effects can be achieved.

What is claimed is:

1. An image pickup apparatus comprising:
an image pickup module in which an image pickup devices is laminated;
a relay board having a plurality of flying leads which are electrically connected to a proximal end face of the image pickup module through ultrasonic wire bonding; and
a plurality of cables electrically connected to a plurality of connection lands of the relay board, wherein
a bending portion is formed on the relay board so that the plurality of cables are fitted within a projection surface of the proximal end face of the image pickup module, and
the bending portion is bent so as to have an arched cross section.

2. The image pickup apparatus according to claim 1, wherein the relay board comprises:
a small-width portion on a distal end side connected to the image pickup module; and
a wide large-width portion comprising the bending portion on a proximal end side of the small-width portion and provided with the plurality of connection lands, and
core wires of the plurality of cables are electrically connected to the plurality of connection lands by solder.

3. The image pickup apparatus according to claim 2, wherein
cables of large diameter of the plurality of cables are connected to a center of the relay board and cables of small diameter are connected to a side part.

4. The image pickup apparatus according to claim 3, wherein the cables of large diameter and the cables of small diameter are connected to a same side of the relay board.

5. The image pickup apparatus according to claim 4, wherein the cables of large diameter and the cables of small diameter are connected to an inside surface of the relay board bent by the bent portion.

6. The image pickup apparatus according to claim 3, wherein the cables of large diameter are connected to an inside surface of the relay board bent by the bending portion and the cables of small diameter are connected to an outside surface of the relay board bent by the bending portion.

7. The image pickup apparatus according to claim 6, wherein notch portions are formed in the relay board from both sides of a boundary portion between the small-width portion and the large-width portion.

8. The image pickup apparatus according to claim 7, wherein a plurality of slits are formed in a front-rear direction of the large-width portion.

9. The image pickup apparatus according to claim 3, wherein
the cables of large diameter are coaxial cables, and
the bending portion is formed at the large-width portion along an external conductor of the coaxial cables.

10. The image pickup apparatus according to claim 2, wherein
the relay board comprises two arm portions that extend toward a distal end side on both side portions of the large-width portion and are in contact with the proximal end face of the image pickup module, and
holes for a jig to hold the relay board are formed in the two arm portions.

11. An image pickup apparatus comprising:
an image pickup module in which an image pickup device is laminated;
a relay board having a plurality of flying leads which are electrically connected to a proximal end face of the image pickup module through ultrasonic wire bonding; and
a plurality of cables electrically connected to a plurality of connection lands of the relay board, wherein
the relay board comprises:
a small-width portion on a distal end side connected to the image pickup module; and
a wide large-width portion comprising the bending portion on a proximal end side of the small-width portion and provided with the plurality of connection lands, the bending portion is formed on the relay board so that the plurality of cables are fitted within a projection surface of the proximal end face of the image pickup module, and
cables of large diameter of the plurality of cables are coaxial cables, and
the bending member along an external conductor of the coaxial cables is formed at the large-width portion.

12. An endoscope apparatus comprising:
the image pickup apparatus according to claim 1; and
an insertion portion comprising a distal end portion in which the image pickup apparatus is incorporated.

13. A method of electrical connection to an image pickup module in the image pickup apparatus according to claim 1, the method comprising:
soldering the plurality of cables to the plurality of connection lands of the relay board;
ultrasonically wire bonding a plurality of flying leads of the relay board to a plurality of terminals provided on the proximal end face of the image pickup module; and
forming the bending member on the relay board so that the plurality of cables are fitted within the projection surface of the proximal end face of the image pickup module.
